(19) United States
(12) Patent Application Publication
(10) Pub. No.: US 2004/0223938 A1
(43) Pub. Date: Nov. 11, 2004

(54) Hair conditioning composition comprising polysorbates

(76) Inventors: Yujun Li, Kobe (JP); Jian-Zhong Yang, Kobe (JP); Jun Hasegawa, Kobe (JP)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/825,878

(22) Filed: Apr. 16, 2004

Related U.S. Application Data
(60) Provisional application No. 60/463,562, filed on Apr. 17, 2003.

(51) Int. Cl. A61K 7/06; A61K 7/11; A61K 7/075; A61K 7/08

(52) U.S. Cl. 424/70.13; 424/70.28

(57) ABSTRACT

Disclosed is a hair conditioning composition comprising by weight: (a) 0.01-10% of a mono-long alkyl quaternized ammonium salt cationic surfactant; (b) 2-20% of a high melting point fatty compound; (c) 0.0014% of a polysorbate; and (d) an aqueous carrier. The hair conditioning composition of the present invention can provide clean feel during and after rinsing the hair, while providing improved conditioning benefits to the hair.

### CROSS REFERENCE TO RELATED APPLICATION

This application claims the benefit of U.S. Provisional Application No. 60/463,562, filed on Apr. 17, 2003.

### FIELD OF INVENTION

The present invention relates to a hair conditioning composition comprising a cationic surfactant, a high melting point fatty compound, a polysorbate, and an aqueous carrier. The composition of the present invention can provide clean feel during and after rinsing while providing improved conditioning benefits.

### BACKGROUND OF THE INVENTION

A variety of approaches have been developed to condition the hair. A common method of providing conditioning benefit is through the use of conditioning agents such as cationic surfactants and polymers, high melting point fatty compounds, low melting point oils, silicone compounds, and mixtures thereof. Most of these conditioning agents are known to provide various conditioning benefits. For example, some cationic surfactants, when used together with some high melting point fatty compounds, are believed to provide a gel matrix which is suitable for providing a variety of conditioning benefits such as softness and moisturized feel on dry hair.

Although a variety of conditioning compositions have been developed, there is still a need for conditioner compositions which provide improved conditioning benefits. A common method of providing improved conditioning benefits is the addition of increased level of conditioning agents such as high melting point fatty compound. However, conditioner compositions containing such increased level of conditioning ingredients also provide long-lasting slimy feel during rinsing the hair, and sticky and greasy feel even after rinsing the hair.

For consumers who prefer clean feel, such feels are not desirable. One common method of obtaining clean feel is rinsing the hair by a large amount of water. However, such rinsing activity provides less deposition of conditioning agents on the hair, thus less conditioning benefits to the hair. Thus, there is a need for a conditioner composition which provides improved clean feel during and after rinsing the hair, so that consumers can easily leave the hair and/or hands with a clean feel while depositing sufficient amount of the conditioning agents on the hair.

Based on the foregoing, there remains a need for conditioning compositions which provide improved clean feel during and after rinsing the hair, while providing improved conditioning benefits such as softness and moisturized feel on dry hair.

None of the existing art provides all of the advantages and benefits of the present invention.

### SUMMARY OF THE INVENTION

The present invention is directed to a hair conditioning composition comprising by weight:

(a) from about 0.1% to about 10% of a cationic surfactant having a formula:

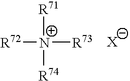

wherein one of R⁷¹, R⁷², R⁷³ and R⁷⁴ is selected from an aliphatic group of from 12 to 30 carbon atoms or an aromatic, alkoxy, polyoxyalkylene, alkylamido, hydroxyalkyl, aryl or alkylaryl group having up to about 22 carbon atoms; the remainder of R⁷¹, R⁷², R⁷³ and R⁷⁴ are independently selected from an aliphatic group of from 1 to about 8 carbon atoms or an aromatic, alkoxy, polyoxyalkylene, alkylamido, hydroxyalkyl, aryl or alkylaryl group having up to about 8 carbon atoms; and X⁻ is a salt-forming anion such as those selected from halogen, acetate, citrate, lactate, glycolate, phosphate, nitrate, sulfonate, sulfate, alkylsulfate, and alkyl sulfonate radicals;

(b) from about 2% to about 20% of a high melting point fatty compound;

(c) from about 0.001% to about 4% of a polysorbate having a formula:

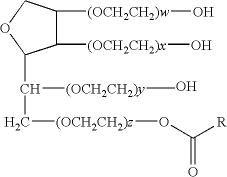

wherein w, x, and y is respectively an integer of 0-20, and z is an integer of 1-20, and wherein w+x+y+z=4-30; and R is a linear or branched, and saturated or unsaturated alkyl having 11-23 carbon atoms; and

(d) an aqueous carrier.

The conditioning composition of the present invention can provide improved clean feel during and after rinsing the hair, while providing improved conditioning benefits such as softness and moisturized feel on dry hair.

These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

Cationic Surfactant

The compositions of the present invention comprise a cationic surfactant. The cationic surfactant is a mono-long alkyl quaternized ammonium salt having the formula (I):

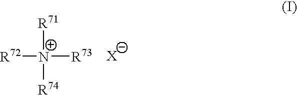

wherein one of R⁷¹, R⁷², R⁷³ and R⁷⁴ is selected from an aliphatic group of from 12 to 30 carbon atoms or an aromatic, alkoxy, polyoxyalkylene, alkylamido, hydroxyalkyl, aryl or alkylaryl group having up to about 22 carbon atoms; the remainder of R⁷¹, R⁷², R⁷³ and R⁷⁴ are independently selected from an aliphatic group of from 1 to about 8 carbon atoms or an aromatic, alkoxy, polyoxyalkylene, alkylamido, hydroxyalkyl, aryl or alkylaryl group having up to about 8 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The aliphatic groups can contain, in addition to carbon and hydrogen atoms, ether linkages, and other groups such as amino groups. The longer chain aliphatic groups, e.g., those of about 12 carbons, or higher, can be saturated or unsaturated. Preferably, one of R⁷¹, R⁷², R⁷³ and R⁷⁴ is selected from an alkyl group of from 12 to 30 carbon atoms, more preferably from 16 to 22 carbon atoms, still more preferably from 18 to 22 carbon atoms, even more preferably 22 carbon atoms; the remainder of R⁷¹, R⁷², R⁷³ and R⁷⁴ are independently selected from CH₃, C₂H₅, C₂H₄₀H, CH₂C₆H₅, and mixtures thereof; and X is selected from the group consisting of Cl, Br, CH₃OSO₃, and mixtures thereof. It is believed that such mono-long alkyl quaternized ammonium salts can provide improved clean feel, compared to multi-long alkyl quaternized ammonium salts. It is also believed that mono-long alkyl quaternized ammonium salts can provide improved conditioning benefits, especially improved softness and improved moisturized feel on dry hair, compared to amine or amine salt cationic surfactants.

Nonlimiting examples of such mono-long alkyl quaternized ammonium salt cationic surfactants include: behenyl trimethyl ammonium chloride available, for example, with tradename Genamine KDMP from Clariant, with tradename INCROQUAT TMC-80 from Croda and ECONOL TM22 from Sanyo Kasei; stearyl trimethyl ammonium chloride available, for example, with tradename CA-2450 from Nikko Chemicals; cetyl trimethyl ammonium chloride available, for example, with tradename CA-2350 from Nikko Chemicals; hydrogenated tallow alkyl trimethyl ammonium chloride; stearyl dimethyl benzyl ammonium chloride; and stearoyl amidopropyl dimethyl benzyl ammonium chloride.

Among them, more preferred cationic surfactants are those having a longer alkyl group, i.e., C18-22 alkyl group. Such cationic surfactants include, for example, behenyl trimethyl ammonium chloride and stearyl trimethyl ammonium chloride, and still more preferred is behenyl trimethyl ammonium chloride. It is believed that; cationic surfactants having a longer alkyl group provide improved deposition on the hair, thus can provide improved conditioning benefits such as improved softness on dry hair, compared to cationic surfactant having a shorter alkyl group. It is also believed that such cationic surfactants can provide reduced irritation, compared to cationic surfactants having a shorter alkyl group.

The cationic surfactant is included in the composition at a level by weight of preferably from about 0.1% to about 10%, more preferably from about 1% to about 8%, still more preferably from about 2% to about 5%, in view of providing improved conditioning benefits such as softness and moisturized feel on dry hair.

The cationic surfactant, together with below high melting fatty compound, and an aqueous carrier, provides a gel matrix which is suitable for providing various conditioning benefits such as slippery fool on wet hair and softness and moisturized feel on dry hair. In view of providing the above gel matrix, the cationic surfactant and the high melting point fatty compound are contained at a level such that the mole ratio of the cationic surfactant to the high melting point fatty compound is in the range of, preferably from about 1:1 to 1:10, more preferably from about 1:2 to 1:6.

High Melting Point Fatty Compound

The hair conditioning composition of the present invention comprises a high melting point fatty compound. The high melting point fatty compound is included in the composition at a level of preferably from about 2% to about 20%, more preferably from about 3% to about 15%, still more preferably from about 4% to about 8% by weight of the composition, in view of providing improved conditioning benefits such as softness and moisturized feel on dry hair.

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about 22 carbon atoms. These fatty alcohols are saturated and can be straight or branched chain alcohols. Preferred fatty alcohols include, for example, cetyl alcohol, stearyl alcohol, behenyl alcohol, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

Commercially available high melting point fatty compounds useful herein include: cetyl alcohol, stearyl alcohol, and behenyl alcohol having tradenames KONOL series available from Shin Nihon Rika (Osaka, Japan), and NAA series available from NOF (Tokyo, Japan); pure behenyl alcohol having tradename 1-DOCOSANOL available from WAKO (Osaka, Japan).

Polysorbate

The composition of the present invention comprises a polysorbate.

Polysorbates are mono-esters those having a formula:

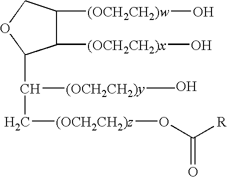

wherein w, x, and y is respectively an integer of 0-20, preferably 1-20, and z is an integer of 1-20, and wherein w+x+y+z=4-30, preferably 4-25, more preferably 10-25; and R is a linear or branched, and saturated or unsaturated alkyl having 11-23 carbon atoms, preferably from 12 to 18 carbon atoms.

The inventors of the present invention have found that; when used in the above gel matrix, poylsorbates can provide improved clean feel during and after rinsing the hair, so that consumers can easily leave the hair and/or hands with a clean feel, while providing improved conditioning benefits such as softness and moisturized feel on dry hair.

Polysorbates have several oxyethylene and/or polyoxyethylene portions. It is believed that, by these portions, polysorbate can provide the above improved benefits, compared to other nonionic surfactants having no or less oxyethylene and/or polyoxyethylene portions. Polysorbates useful herein have a HLB value of preferably 10 or more, more preferably 13 or more, in view of providing improved clean feel.

Polysorbates especially useful herein include, for example, polysorbate-20 (POE(20) sorbitan monolaurate) having HLB value of 16.7, polysorbate-21 (POE(4)sorbitan monolaurate) having HLB value of 13.3, polysorbate-40 (POE(20)sorbitan monopalmitate) having HLB value of 15.6, polysorbate-60 (POE(20)sorbitan monostearate) having HLB value of 14.9, polysorbate-61 (POE(4)sorbitan monostearate) having HLB value of 9.6, polysorbate-80 (POE(20)sorbitan monooleate) having HLB value of 15.0, and polysorbate-81 (POE(4)sorbitan monooleate) having HLB value of 10.0.

Polysorbates are included in the composition at a level by weight of preferably from about 0.001% to about 4%, more preferably from about 0.005% to about 2%, still more preferably from about 0.01% to about 0.4%, even more preferably from about 0.01% to about 0.1%, in view of providing improved clean feel during and after rinsing the hair, while maintaining conditioning benefits such as softness and moisturized feel on the dry hair.

In view of providing improved clean feel, the weight ratio of the polysorbate to the total of the cationic surfactant and the high melting point fatty compound is preferably from about 1:10 to about 1:500, more preferably from about 1:25 to about 1:400, still more preferably from about 1:30 to about 1:300.

Aqueous Carrier

The conditioning composition of the present invention comprises an aqueous carrier. The level and species of the carrier are selected according to the compatibility with other components, and other desired characteristic of the product.

The carrier useful in the present invention includes water and water solutions of lower alkyl alcohols and polyhydric alcohols. The lower alkyl alcohols useful herein are monohydric alcohols having 1 to 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product. Generally, the compositions of the present invention comprise from about 20% to about 99%, preferably from about 30% to about 95%, and more preferably from about 80% to about 95% water.

Silicone Compound

Preferably, the compositions of the present invention preferably contain a silicone compound. It is believed that the silicone compound can provide smoothness and softness on dry hair. The silicone compounds may further be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and mixtures thereof. Commercially available silicone emulsions useful herein include, for example, dimethicone emulsion wherein the dimethicone has a viscosity of 100,000 mPa·s with a tradename BY22-067R available from Dow Corning.

The silicone compounds herein are preferably used at levels by weight of the composition of from about 0.1% to about 20%, more preferably from about 0.5% to about 10%, still more preferably from about 1% to about 8%.

The silicone compounds useful herein, as a single compound, or as a blend or mixture of at least two compounds, have a viscosity of preferably from about 1,000 to about 2,000,000 mPa·s at 25° C.

The viscosity can be measured by means of a glass capillary viscometer as set forth in Dow Corning Corporate Test Method CTM0004, Jul. 20, 1970. Suitable silicone fluids include polyalkyl siloxanes, polyaryl siloxanes, polyalkylaryl siloxanes, polyether siloxane copolymers, amino substituted silicones, quaternized silicones, and mixtures thereof. Other nonvolatile silicone compounds having conditioning properties can also be used.

The silicone compounds useful herein include polyalkyl or polyaryl siloxanes with the following structure:

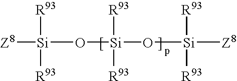

wherein R⁹³ is alkyl or aryl, and p is an integer from about 7 to about 8,000. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the hair, is compatible with the other components of the composition, is chemically stable under normal use and storage conditions, and is capable of being deposited on and conditions the hair. Suitable Z⁸ groups include hydroxy, methyl, methoxy, ethoxy, propoxy, and aryloxy. The two R⁹³ groups on the silicon atom may represent the same group or different groups. Preferably, the two R⁹³ groups represent the same group. Suitable R⁹³ groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their Viscasil® and TSF 451 series, and from Dow Corning in their Dow Corning SH200 series.

The above polyalkylsiloxanes are available, for example, as a mixture with silicone compounds having a lower viscosity. Such mixtures have a viscosity of preferably from about 1,000 mPa·s to about 100,000 mPa·s, more preferably from about 5,000 mPa·s to about 50,000 mPa·s. Such mixtures preferably comprise: (i) a first silicone having a viscosity of from about 100,000 mPa·s to about 1,000,000 mPa·s at 25° C., preferably from about 200,000 mPa·s to about 1,000,000 mPa·s; and (ii) a second silicone having a viscosity of from about 1 mPa·s to about 10,000 mPa·s at 25° C., preferably from about 5 mPa·s to about 5,000 mPa·s. Such mixtures useful herein include, for example, a blend of dimethicone having a viscosity of 500,000 mPa·s and dimethicone having a viscosity of 200 mPa·s available from GE Toshiba, and a blend of dimethicone having a viscosity of 500,000 mPa·s and cyclopentasiloxane available from GE Toshiba

The silicone compounds useful herein also include a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. The silicone gums are available, for example, as a mixture with silicone compounds having a lower viscosity. Such mixtures useful herein include, for example, Gum/Cyclomethicone blend available from Shin-Etsu.

The silicone compounds that can be used include, for example, a polypropylene oxide modified polydimethylsiloxane although ethylene oxide or mixtures of ethylene oxide and propylene oxide can also be used. The ethylene oxide and polypropylene oxide level should be sufficiently low so as not to interfere with the dispersibility characteristics of the silicone. These materials are also known as dimethicone copolyols.

Silicone compounds useful herein also include amino substituted materials. Suitable alkylamino substituted silicone compounds include those represented by the following structure:

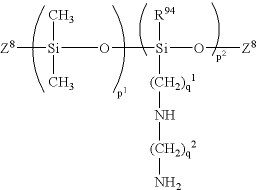

wherein R⁹⁴ is H, CH₃ or OH; p¹ and p² are integers of 1 or above, and wherein sum of p¹ and p² is from 650 to 1,500; q¹ and q² are integers of from 1 to 10. Z⁸ represents groups which block the ends of the silicone chains. Suitable Z⁸ groups include hydroxy, methyl, methoxy, ethoxy, propoxy, and aryloxy. Highly preferred are those known as “amodimethicone”. Commercially available amodimethicones useful herein include, for example, BY16-872 available from Dow Corning, and ADM1100 available from Wacker.

Other amino substituted silicone polymers which can be used are represented by the formula:

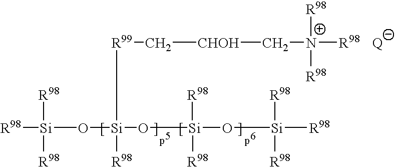

where R⁹⁸ denotes a monovalent hydrocarbon radical having from 1 to 18 carbon atoms, preferably an alkyl or alkenyl radical such as methyl; R⁹⁹ denotes a hydrocarbon radical, preferably a C₁-C₁₈ alkylene radical or a C₁-C₁₈, and more preferably C₁-C₈, alkyleneoxy radical; Q⁻ is a halide ion, preferably chloride; p⁵ denotes an average statistical value from 2 to 20, preferably from 2 to 8; p⁶ denotes an average statistical value from 20 to 200, and preferably from 20 to 50. A preferred polymer of this class is available from Union Carbide under the name “UCAR SILICONE ALE 56.”

Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; emollients such as PPG-3 myristyl ether with tradename Varonic APM available from Goldschmidt, Trimethyl pentanol hydroxyethyl ether, PPG-11 stearyl ether with tradename Varonic APS available from Goldschmidt, Stearyl heptanoate with tradename Tegosoft SH available from Goldschmidt, Lactil (mixture of Sodium lactate, Sodium PCA, Glycine, Fructose, Urea, Niacinamide, Inositol, Sodium Benzoate, and Lactic acid) available from Goldschmidt, Ethyl hexyl palmitate with tradename Saracos available from Nishin Seiyu and with tradename Tegosoft OP available from Goldschmidt; hair-fixative polymers such as amphoteric fixative polymers, cationic fixative polymers, anionic fixative polymers, nonionic fixative polymers, and silicone grafted copolymers; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; hair oxidizing (bleaching) agents, such as hydrogen peroxide, perborate and persulfate salts; hair reducing agents such as the thioglycolates; perfumes; and sequestering agents, such as disodium ethylenediamine tetra-acetate; ultraviolet and infrared screening and absorbing agents such as octyl salicylate; and antidandruff agents such as zinc pyrithione and salicylic acid.

Polypropylene Glycol

Polypropylene glycol useful herein are those having a weight average molecular weight of from about 200 g/mol to about 100,000 g/mol, preferably from about 1,000 g/mol to about 60,000 g/mol. Without intending to be limited by theory, it is believed that the polypropylene glycol herein deposits onto, or is absorbed into hair to act as a moisturizer buffer, and/or provides one or more other desirable hair conditioning benefits.

The polypropylene glycol useful herein may be either water-soluble, water-insoluble, or may have a limited solubility in water, depending upon the degree of polymerization and whether other moieties are attached thereto. The desired solubility of the polypropylene glycol in water will depend in large part upon the form (e.g., leave-on, or rinse-off form) of the hair care composition. For example, a rinse-off hair care composition, it is preferred that the polypropylene glycol herein has a solubility in water at 25° C. of less than about 1 g/100 g water, more preferably a solubility in water of less than about 0.5 g/100 g water, and even more preferably a solubility in water of less than about 0.1 g/100 g water.

The polypropylene glycol can be included in the hair conditioning composition of the present invention at a level of, preferably from about 0.01% to about 10%, more preferably from about 0.05% to about 6%, still more preferably from about 0.1% to about 3% by weight of the composition.

Low Melting Point Oil

Low melting point oils useful herein are those having a melting point of less than 25° C. The low melting point oil useful herein is selected from the group consisting of: hydrocarbon having from 10 to about 40 carbon atoms; unsaturated fatty alcohols having from about 10 to about 30 carbon atoms such as oleyl alcohol; unsaturated fatty acids having from about 10 to about 30 carbon atoms; fatty acid derivatives; fatty alcohol derivatives; ester oils such as pentaerythritol ester oils, trimethylol ester oils, citrate ester oils, and glyceryl ester oils; poly α-olefin oils; and mixtures thereof. Preferred low melting point oils herein are selected from the group consisting of: ester oils such as pentaerythritol ester oils, trimethylol ester oils, citrate ester oils, and glyceryl ester oils; poly α-olefin oils; and mixtures thereof,

Particularly useful pentaerythritol ester oils and trimethylol ester oils herein include pentaerythritol tetraisostearate, pentaerythritol tetraoleate, trimethylolpropane triisostearate, trimethylolpropane trioleate, and mixtures thereof. Such compounds are available from Kokyo Alcohol with tradenames KAKPTI, KAKTTI, and Shin-nihon Rika with tradenames PTO, ENUJERUBU TP3SO.

Particularly useful citrate ester oils herein include triisocetyl citrate with tradename CITMOL 316 available from Bemel, triisostearyl citrate with tradename PELEMOL TISC available from Phoenix, and trioctyldodecyl citrate with tradename CITMOL 320 available from Bemel.

Particularly useful glyceryl ester oils herein include triisostearin with tradename SUN ESPOL G-318 available from Taiyo Kagaku, triolein with tradename CITHROL GTO available from Croda Surfactants Ltd., trilinolein with tradename EFADERMA-F available from Vevy, or tradename EFA-GLYCERIDES from Brooks.

Particularly useful poly α-olefin oils herein include polydecenes with tradenames PURESYN 6 having a number average molecular weight of about 500 and PURESYN 100 having a number average molecular weight of about 3000 and PURESYN 300 having a number average molecular weight of about 6000 available from Exxon Mobil Co.

Cationic Conditioning Polymer

Cationic conditioning polymers useful herein are those having an average molecular weight of at least about 5,000, typically from about 10,000 to about 10 million, preferably from about 100,000 to about 2 million.

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. Other suitable spacer monomers include vinyl esters, vinyl alcohol (made by hydrolysis of polyvinyl acetate), maleic anhydride, propylene glycol, and ethylene glycol. Other suitable cationic polymers useful herein include, for example, cationic celluloses, cationic starches, and cationic guar gums.

Polyethylene Glycol

Polyethylene glycol can also be used as an additional component. The polyethylene glycols useful herein that are especially preferred are PEG-2M wherein n has an average value of about 2,000 (PEG-2M is also known as Polyox WSR® N-10 from Union Carbide and as PEG-2,000); PEG-5M wherein n has an average value of about 5,000 (PEG-5M is also known as Polyox WSR® N-35 and as Polyox WSR® N-80, both from Union Carbide and as PEG-5,000 and Polyethylene Glycol 300,000); PEG-7M wherein n has an average value of about 7,000 (PEG-7M is also known as Polyox WSR® N-750 from Union Carbide); PEG-9M wherein n has an average value of about 9,000 (PEG-9M is also known as Polyox WSR® N-3333 from Union Carbide); and PEG-14M wherein n has an average value of about 14,000 (PEG-14M is also known as Polyox WSR® N-3000 from Union Carbide).

Compositions

The conditioning composition of the present invention is preferably substantially free of anionic surfactants and anionic polymers, in view of stability of the gel matrix. In the present invention, “substantially free of anionic surfactants and anionic polymers” means that the composition contain 1% or less, preferably 0.5% or less, more preferably totally 0% of total of anionic surfactants and anionic polymers.

Product Forms

The conditioning compositions of the present invention can be in the form of rinse-off products or leave-on products, can be transparent or opaque, and can be formulated in a wide variety of product forms, including but not limited to creams, gels, emulsions, mousses and sprays. The conditioning composition of the present invention is especially suitable for rinse-off hair conditioner.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

[Compositions]

| Components | Ex. 1 | Ex. 2 | Ex. 3 | Ex. 4 | Ex. 5 | Ex. 6 | Ex. 7 | Ex. 8 |
|---|---|---|---|---|---|---|---|---|
| Behentrimonium chloride*1 | 3.38 | 3.38 | 3.38 | 2.0 | 4.05 | — | 2.0 | 3.38 |
| Stearyl trimethyl ammonium | — | — | — | — | — | 3.5 | 2.0 | — |
| chloride*2 | | | | | | | | |
| Cetyl alcohol*3 | 2.32 | 2.32 | 2.32 | 2.0 | 2.5 | 2.3 | 2.0 | 2.32 |
| Stearyl alcohol*4 | 4.18 | 4.18 | 4.18 | 3.0 | 4.5 | 4.2 | — | 4.18 |
| Behenyl alcohol*5 | — | — | — | — | — | — | 4.0 | — |
| Polysorbate-60*6 | 0.2 | 0.2 | 0.05 | 0.025 | 0.1 | — | — | 0.05 |
| Polysorbate-20*7 | — | — | — | — | — | 0.05 | 0.05 | — |
| Polysorbate-80*8 | — | — | — | — | — | — | 0.2 | — |
| Dimethicone blend*9 | 4.2 | — | 1.0 | 5.0 | — | 2.0 | 4.2 | 4.2 |
| Dimethicone/Cyclomethicone*10 | — | 4.2 | — | — | 4.2 | — | — | — |
| Aminosilicone*11 | — | — | — | — | — | — | 0.5 | — |
| PPG-34*12 | — | — | — | — | — | — | — | 0.5 |
| Poly-α-olefin oil*13 | — | — | — | — | — | — | — | 1.0 |
| Disodium EDTA | 0.13 | 0.13 | 0.13 | 0.13 | 0.13 | 0.13 | 0.13 | 0.13 |
| Benzyl alcohol | 0.4 | 0.4 | 0.4 | 0.4 | 0.4 | 0.4 | 0.4 | 0.4 |
| Kathon CG*14 | 0.033 | 0.033 | 0.033 | 0.033 | 0.033 | 0.033 | 0.033 | 0.033 |
| Perfume | 0.25 | 0.25 | 0.25 | 0.25 | 0.25 | 0.25 | 0.25 | 0.25 |
| Panthenol*15 | 0.05 | 0.05 | 0.05 | — | 0.05 | 0.05 | 0.05 | 0.05 |
| Panthenyl ethyl ether*16 | 0.05 | 0.05 | 0.05 | — | 0.05 | 0.05 | 0.05 | 0.05 |
| Hydrolyzed collagen*17 | — | — | — | — | 0.01 | 0.01 | 0.01 | 0.01 |
| Vitamin E*18 | — | — | — | — | 0.01 | 0.01 | 0.01 | 0.01 |
| Octyl methoxycinnamate | — | — | — | — | 0.09 | 0.09 | 0.09 | 0.09 |
| Benzophenone-3 | — | — | — | — | 0.09 | 0.09 | 0.09 | 0.09 |

| Deionized Water | q.s. to 100% | | | | | | | | |
|---|---|---|---|---|---|---|---|---|---|
| Components | Ex. 9 | Ex. 10 | Ex. 11 | Ex. 12 | Ex. 13 | Ex. 14 | Ex. 15 | Ex. 16 | Ex. 17 |
| Behentrimonium chloride*1 | 3.38 | 3.38 | 3.45 | 2.25 | 2.25 | 2.25 | 2.25 | 3.38 | 4.05 |
| Cetyl alcohol*3 | 2.32 | 2.32 | 1.97 | 1.86 | 2.32 | 1.86 | 1.86 | 2.32 | 2.5 |
| Stearyl alcohol*4 | 4.18 | 4.18 | 3.55 | 4.64 | 4.18 | 4.64 | 4.64 | 4.18 | 4.5 |
| Polysorbate-60*6 | 0.2 | — | 0.1 | 0.2 | 0.2 | 0.2 | 0.2 | 0.2 | 0.1 |
| Polysorbate-20*7 | — | 0.2 | 0.1 | — | — | — | — | — | — |
| Dimethicone blend*9 | — | 4.2 | — | 4.2 | — | 7.5 | — | — | — |
| Dimethicone blend*19 | — | — | 4.2 | — | — | — | — | — | — |
| Dimethicone/Cyclomethicone*20 | — | — | — | — | — | — | — | 4.2 | 4.2 |
| Aminosilicone*21 | 2.0 | 0.5 | — | 0.5 | 2.0 | — | — | — | — |
| Aminosilicone*22 | — | — | — | — | — | — | 3.5 | — | — |
| C13-C16 Isoparaffin*23 | — | — | — | — | — | — | 1.5 | — | — |
| Disodium EDTA | 0.13 | 0.13 | 0.13 | 0.13 | 0.13 | 0.13 | 0.13 | 0.13 | 0.13 |
| Benzyl alcohol | 0.4 | 0.4 | 0.4 | 0.4 | 0.4 | 0.4 | 0.4 | 0.4 | 0.4 |
| Kathon CG*14 | 0.033 | 0.033 | 0.033 | 0.033 | 0.033 | 0.033 | 0.033 | 0.033 | 0.033 |
| Perfume | 0.50 | 0.50 | 0.35 | 0.50 | 0.50 | 0.50 | 0.50 | 0.35 | 0.50 |
| Panthenol*15 | 0.05 | 0.05 | 0.05 | 0.05 | 0.05 | 0.05 | 0.05 | 0.05 | 0.05 |
| Panthenyl ethyl ether*16 | 0.05 | 0.05 | 0.05 | 0.05 | 0.05 | 0.05 | 0.05 | 0.05 | 0.05 |

| | |
|---|---|
| Deionized Water | q.s. to 100% |

Definitions of Components
*1Behentrimonium chloride: Genamin KDMP available from Clariant
*2Steary trimethyl ammonium chloride: CA-2450 available from Nikko Chemicals
*3Cetyl alcohol: Konol series available from Shin Nihon Rika.
*4Stearyl alcohol: Konol series available from Shin Nihon Rika.
*5Behenyl alcohol: Konol series available from Shin Nihon Rika
*6Polysorbate-60: Tween 60 available from ICI
*7Polysorbate-20: Tween 20 available from ICI
*8Polysorbate-80: Tween 80 available from ICI
*9Dimethicone blend: a blend of dimethicone having a viscosity of 500,000 mPa · s and dimethicone having a viscosity of 200 mPa · s available from GE Toshiba
*10Dimethicone/Cyclomethicone: a blend dimethicone having a viscosity of 500,000 mPa · s and cyclopentasiloxane available from GE Toshiba
*11Aminosilicone: BY16-872 available from Dow Corning
*12PPG-34: New Pol PP-2000 available from Sanyo Kasei.
*13Poly-α-olefin oil: Puresyn 100 available from Exxon Mobil
*14Kathon CG: Available from Rohm&Haas
*15Panthenol: Available from Roche.
*16Panthenyl ethyl ether: Available from Roche.
*17Hydrolyzed collagen: Peptein 2000 available from Hormel.
*18Vitamin E: Emix-d available from Eisai.
*19Dimethicone blend: a blend of dimethicone having a viscosity of 18,000,000 mPa · s and dimethicone having a viscosity of 200 mPa · s available from GE Toshiba
*20Dimethicone/Cyclomethicone: a blend dimethicone having a viscosity of 18,000,000 mPa · s and cyclopentasiloxane available from GE Toshiba
*21Aminosilicone: ADM1100 available from Wacker
*22Aminosilicone: BX3083-1 available from GE Toshiba
*23C13-C16 isoparaffine: Isofol 400 available from Nisseki

Method of Preparation

The conditioning compositions of “Ex. 1” through “Ex. 17” as shown above can be prepared by any conventional method well known in the art. They are suitably made as follows:

Cationic surfactants and high melting point fatty compounds are added to water with agitation, and heated to about 80° C. The mixture is cooled down to about 55° C. Polysorbate can be added to the mixture with agitation at about 55° C., or prior to the cooling down (i.e. at about 80° C.). If included, silicone compounds, perfumes, preservatives are added to the mixture with agitation. Then the mixture is cooled down to room temperature.

Examples 1 through 17 are hair conditioning compositions of the present invention which are particularly useful for rinse-off use. The embodiments disclosed and represented by the previous “Ex. 1” through “Ex. 17” have many advantages. For example, they can provide improved clean feel during and after rinsing the hair, so that consumers can easily leave the hair and/or hands with a clean feel, while providing improved conditioning benefits such as softness and moisturized feel on dry hair.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is, therefore, intended to cover in the appended claims all such changes and modifications that are within the scope of this invention.

What is claimed is:

1. A hair conditioning composition comprising by weight:
(a) from about 0.1% to about 10% of a cationic surfactant having a formula: 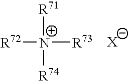
wherein one of R⁷¹, R⁷², R⁷³ and R⁷⁴ is selected from an aliphatic group of from 12 to 30 carbon atoms or an aromatic, alkoxy, polyoxyalkylene, alkylamido, hydroxyalkyl, aryl or alkylaryl group having up to about 22 carbon atoms; the remainder of R⁷¹, R⁷², R⁷³ and R⁷⁴ are independently selected from an aliphatic group of from 1 to about 8 carbon atoms or an aromatic, alkoxy, polyoxyalkylene, alkylamido, hydroxyalkyl, aryl or alkylaryl group having up to about 8 carbon atoms; and X⁻ is a salt-forming anion such as those selected from halogen, acetate, citrate, lactate, glycolate, phosphate, nitrate, sulfonate, sulfate, alkylsulfate, and alkyl sulfonate radicals;
(b) from about 2% to about 20% of a high melting point fatty compound;
(c) from about 0.001% to about 4% of a polysorbate having a formula: 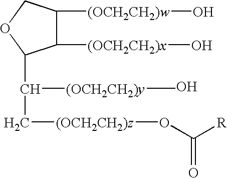
wherein w, x, and y is respectively an integer of 0-20, and z is an integer of 1-20, and wherein w+x+y+z=4-30; and R is a linear or branched, and saturated or unsaturated alkyl having 11-23 carbon atoms; and
(d) an aqueous carrier.

2. The hair conditioning composition of claim 1 wherein the cationic surfactant is water-insoluble.

3. The hair conditioning composition of claim 1 comprising from about 3% to about 15% of the high melting point fatty compound.

4. The hair conditioning composition of claim 1 comprising from about 0.005% to about 2% of the polysorbate.

5. The hair conditioning composition of claim 4 comprising from about 0.01% to about 0.4% of the polysorbate.

6. The hair conditioning composition of claim 1 wherein the polysorbate has a HLB value of 10 or more.

7. The hair conditioning composition of claim 1 wherein the mole ratio of the cationic surfactant to the high melting point fatty compound is from about 1:1 to about 1:10.

8. The hair conditioning composition of claim 1 wherein the weight ratio of the polysorbate to the total of the cationic surfactant and the high melting point fatty compound is from about 1:10 to about 1:500.

9. The conditioning composition of claim 1 further comprising from about 0.1% to about 20% of a silicone compound.

10. The conditioning composition of claim 9, wherein the silicone compound comprises a mixture of a first silicone having a viscosity of from about 100,000 mPa·s to about 1,000,000 mPa·s, and a second silicone having a viscosity of from about 1 mPa·s to about 10,000 mPa·s.
